(19) United States
(12) Patent Application Publication
(10) Pub. No.: US 2016/0220410 A1
(43) Pub. Date: Aug. 4, 2016

(54) CERVICAL BRACE FORMING LORDOSIS

(71) Applicant: Kyu Min Hwang, Palo Alot, CA (US)

(72) Inventor: Kyu Min Hwang, Palo Alto, CA (US)

(73) Assignee: Kyu Min Hwang, Palo Alto, CA (US)

(21) Appl. No.: 15/012,744

(22) Filed: Feb. 1, 2016

(30) Foreign Application Priority Data
Feb. 4, 2015 (KR) 10-2015-0017217

(51) Int. Cl. A61F 5/055

(52) U.S. Cl. CPC A61F 5/055

(57) ABSTRACT

According to the present invention, the cervical brace includes the part of chin support in the form of a circular ring; the part of shoulder support in the form of a circular ring; the hinge part which connects the parts of chin support and shoulder support; the moving part which adjusts the distance between the chin-supporting part and shoulder-supporting part. This moving part corrects the posture of the cervical spine by adjusting height of the chin-supporting part.

The present invention relates to a cervical brace forming lordosis. Specifically, this cervical brace corrects the posture of cervical vertebra by aiding the formation of its normal forward curve.

### BACKGROUND

Human spine is the backbone of human skeletal system that supports the shape of the body. As shown in FIG. 1, the vertebrae column is composed of cervical, thoracic, lumbar, sacral, and coccygeal vertebrae, which correspond to neck bones, back bones, haunch bones, sacrums, and tailbones, respectively.

Looking from the side, a healthy spine has normal lordosis, that is, the two forward curves in cervical and lumbar vertebrae, and normal kyphosis, which is the two backward curves in thoracic and sacral vertebrae. However, damages to centrums, intervertebral discs, and/or surrounding muscles may disrupt this normal shape of spine. When the thoracic curvature is exaggerated or the cervical and lumbar vertebrae have lost their normal forward curvature, it is called Kyphosis (the capitalized terms, Kyphosis and Lordosis, will be used to discuss the abnormal curvatures).

A spine with normal lordosis provides body a physiological and structural support, as the line of gravity runs along the back of the vertebrae and the center of mass is located on the rear side of the spinal vertebrae and joints.

However, if the cervical vertebrae straightens or curves backward, the line of gravity moves towards the front of the vertebrae. This causes the migration of the center of mass to lie above vertebral pulp and the unstable displacement of the centrum. As a result, the vertebral pulp gains tendency to move backwards where the boundary is relatively weaker, increasing the chance to cause cervical herniated nucleus pulposus (HNP), or herniated disc. Thus, it is critical to maintain the normal forward curvature on cervical vertebrae in order to prevent such physiological damage.

As shown above, cervical vertebrae plays an important role in human body.

Therefore, it is urgent to develop a novel cervical brace that not only effectively maintains and/or improves the lordosis but also is straightforward and easy to use.

### SUMMARY

The invention of the lordosis-forming cervical brace, that is, the means to achieve the purposes presented above, is composed of the following: a circular ring-shaped chin-supporting unit; a circular ring-shaped shoulder-supporting unit; two hinge units that connect the chin-supporting and shoulder-supporting units, equipped at both sides of the rear; and control units, located at both sides of the front, which maintain and adjust the gap distance between the two units at the front, connecting the two supporting units. Once the front portion of the chin-supporting unit rotates upward around the aforementioned hinge units, the above-mentioned control units lock up and maintain the gap between the two supporting units.

Also, because the control units are comprised of articulating parts, including interlocking toothed latch wheels, they extend as the chin-supporting unit rotates upward, while the articulating parts interlocks once the chin-supporting unit comes to a halt. When the articulating parts lock up, the distance between the chin- and shoulder-supporting units is fixed and maintained. The chin-supporting unit can rotate downward when the interlocking toothed latch wheels are separated from each other, allowing them to pivot in the opposite direction to reduce the distance between the two supporting units.

Further, the above-mentioned control units can be divided into the upper and lower regulatory arms, centering on the articulating junction. One or more of these arms contain internal springs in order to provide tension when the chin-supporting unit is bearing weight.

In addition, the aforementioned control units may consist of the following: the upper regulatory arm that can assemble and combine with the chin-supporting unit; the first coupling part that is located at the bottom end of the upper arm and includes a toothed latch wheel; the second coupling part that interlocks with the toothed latch wheel of the first coupling part; the lower regulatory arm that is located at the bottom end of the second coupling part and can assemble and combine with the shoulder-supporting unit; the equipped bolt that penetrates the first and second coupling parts; and the compression spring that is implemented around the penetrating bolt to interlock the first and second coupling parts.

Moreover, when the bolt is pressed on its head, the toothed latch wheel of the first coupling part separates from that of the second coupling part, allowing the chin-supporting unit to rotate downward around the hinge units as axis.

Additionally, the abovementioned upper regulatory arm can include the pinning body, where the first coupling part is mounted on the bottom end, as well as the tension-providing body, which contains a spring at its one end. This end of the tension-providing body incorporating the internal spring is inserted into the pinning body to form a complex and exert tension on the chin-supporting unit.

Furthermore, a plate-shaped cushion, or the cervical-supporting part, may be supplemented onto the rear center of the chin- and shoulder-supporting units to provide an extra support on the cervical spine.

Also, the cervical-supporting cushion is enclosed in a covering made out of flexible material. In order to allow an easy use of the device, a small portion of the control units may be exposed out of the covering or the control units may be silhouetted against the covering by tightly fitting the covering around the control units.

### DESCRIPTION OF THE DRAWINGS

FIG. 1 schematically illustrates normal human vertebral column in its correct posture.

FIG. 2 is a perspective view of the lordosis-forming cervical brace.

FIG. 3 is an exploded view of the control units of the invention.

FIG. 4 is a perspective view of the invention when the chin-supporting part is swung upward.

FIG. 5 is a perspective view of the invention from the rear.

FIG. 6 illustrates the rear view of a patient wearing the invention around neck.

FIG. 7 illustrates when a patient first wears the invention around neck.

FIG. 8 illustrates the formation of lordosis achieved when a patient wears the invention by lifting and fixing the chin-supporting part.

FIG. 9 illustrates the patient in FIG. 7 wearing the invention fitted in covering.

FIG. 10 illustrates the patient in FIG. 8 wearing the invention fitted in covering.

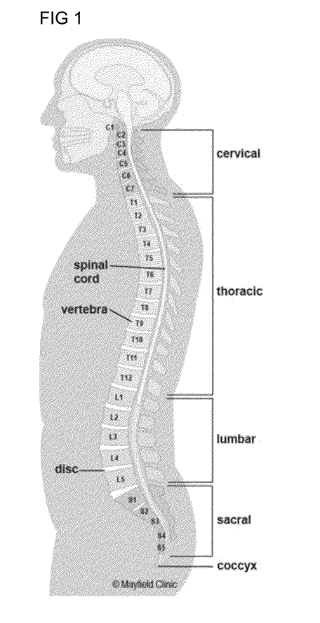

A summary of element numbers used in the drawings is provided below:

| | |
|---|---|
| 10: Chin-supporting part | 20: Shoulder-supporting part |
| 31a, 30b: Hinge part | 40a, 40b: Control units |
| 41: Upper regulatory arm | 42: Pinning part |
| 43: Moving Part | 44: Spring |
| 45: First coupling part | 46, 53: Toothed latch wheels |
| 47, 54: Hole | 51: Lower regulatory arm |
| 52: Second coupling part | 55: Bolt |
| 56: Head | 57: Compression spring |
| 60: Cervical-supporting part | 70: Covering |

100: Lordosis-forming cervical brace

### DESCRIPTION

Hereinafter, the embodiments of the present invention are described in detail with reference to the accompanying drawings.

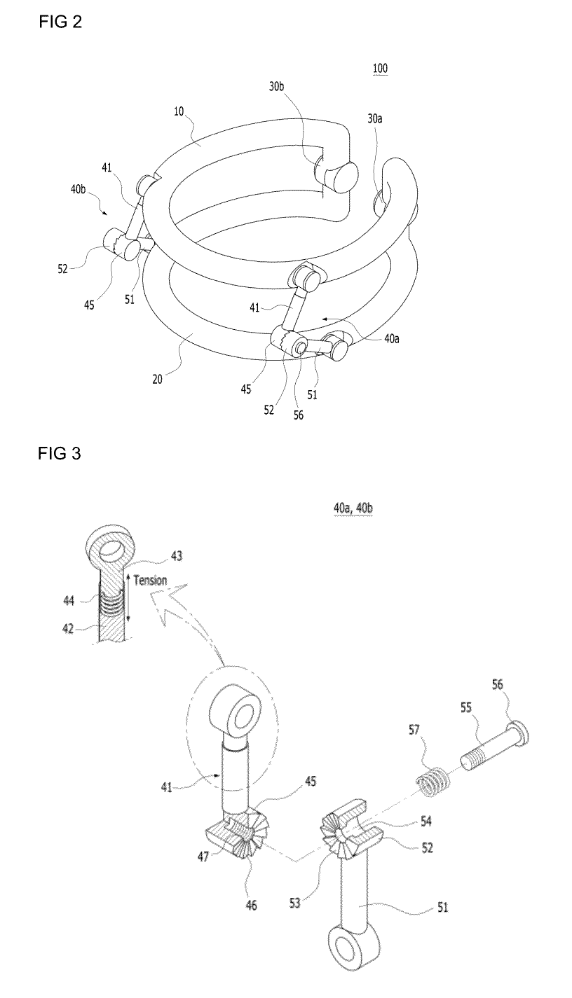

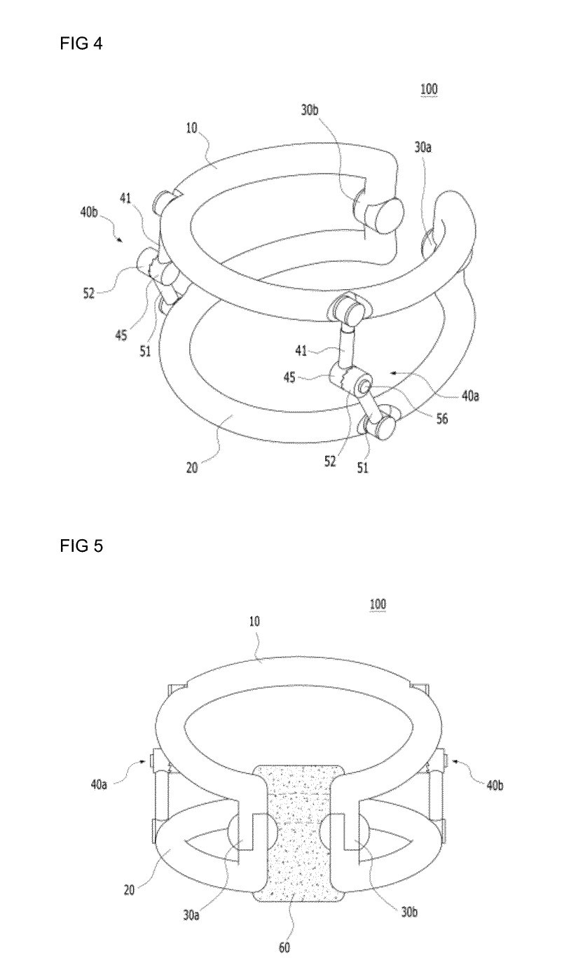

Referring to FIGS. 2 to 4, the lordosis-forming cervical brace consists of a ring-shaped chin-supporting unit 10, a ring-shaped shoulder-supporting unit 20, hinge units 30, and control units 40. The chin-supporting 10 and shoulder-supporting 20 units are preferably made of a flexible metal or plastic material.

The hinge units 30 are equipped in the bent portion of the rear sides of the chin-supporting 10 and shoulder-supporting 20 units, connecting the chin-supporting 10 and shoulder-supporting 20 units. The chin-supporting 10 and shoulder-supporting 20 units can rotate up and down on the hinge units 30. The hinge units (30a, 30b are equipped on each of the rear left and right bent portions.

The control units 40 are located on both left and right sides of the front of the chin-supporting 10 and shoulder-supporting 20 units, connecting the chin-supporting 10 and shoulder-supporting 20 units, maintaining and adjusting the distance between the frontal portions of the chin-supporting 10 and shoulder-supporting 20 units. Two control units 40a, 40b are equipped on each of the frontal left and right sides.

The chin-supporting unit 10 rotates upward around the hinge units 30 to increase the distance between the chin-supporting unit 10 and the shoulder-supporting unit 20. When the chin-supporting unit 10 stops its motion, the interval distance is maintained without additional control. The chin-supporting unit 10 can be rotated upward either by directly lifting the chin-supporting unit 10 or by pivoting the control units 40 to push the chin-supporting unit 10 upward.

The control units 40a, 40b are composed of articulating parts, including toothed latch wheels 46, 53, so that they extend when the chin-supporting unit 10 rotates upward, but lock up when the chin-supporting unit 10 stops moving. When the chin-supporting unit 10 comes to a halt, the articulating parts are fastened as the toothed latch wheels 46, 53 interlock with each other, maintaining the interval distance between the chin-supporting unit 10 and the shoulder-supporting unit 20. The interlocked toothed latch wheels 46, 53 can be separated to allow them to pivot in the opposite direction, rotating the chin-supporting unit 10 downward.

Referring to FIG. 3, the articulating parts indicate the first coupling part 45 and the second coupling part 52, which can be combined and separated by the interaction between the two toothed latch wheels 46, 53. The first coupling part 45 and the second coupling part 52 can only rotate in one direction (Direction 1) when they are combined by elastic coupling of the latch structure and the compression spring 57. When the toothed latch wheels 46, 53 are separated by the user's operation, they can rotate in the opposite direction (Direction 2). As described above, the first coupling part 45 rotates in Direction 1, as the chin-supporting unit 10 is raised upward. This rotation can occur even when the toothed latch wheels 46, 53 are combined together. Once the first coupling part 45 stops its motion, because the structure of the latch structure prevents its rotation to Direction 2, the articulating parts become fixed. When the user presses upon the head 56 of the bolt 55, the toothed latch wheels 46, 53 are separated from each other, and the first coupling part 45 and the second coupling part 52 can rotate in Direction 2, while staying intact with each other due to the presence of the bolt 55.

The control units 40a, 40b are comprised of the upper regulatory arm 41 and the lower regulatory arm 51, centering on the articulating parts 45, 52. Either or both of the upper regulatory arm 41 and lower regulatory arm 51 contain internal springs to provide tension when the chin-supporting unit 10 is bearing weight. Each of the upper regulatory arm 41 and the lower regulatory arm 51 consists of two frames, one of which inserted into the other to form a combinational body. The upper regulatory arm 41 and the lower regulatory arm 51 also include internal springs 44, which can provide tension to absorb shock when bearing weight from the chin-supporting unit 10. The upper regulatory arm 41 is hinged to the chin-supporting unit 10, while the lower regulatory arm 51 is hinged to the shoulder-supporting unit 20.

Moreover, the control units 40a, 40b consist of the upper regulatory arm 41 that can assemble and combine with the chin-supporting unit 10, the first coupling part 45 that is located at the bottom end of the upper regulatory arm 41 and includes a toothed latch wheel 46, the second coupling part 52 of which toothed latch wheel 53 interlocks with the toothed latch wheel 46 of the first coupling part 45, the lower regulatory arm 51 that is located at the bottom end of the second coupling part 52 and can assemble and combine with the shoulder-supporting unit 20, the equipped bolt 55 that penetrates the first coupling part 45 and the second coupling part 52, and the compression spring 57 that is implemented around the penetrating bolt 55 to interlock the first coupling part 45 and the second coupling part 52.

The hole 47 on the first coupling part 45 forms a screw thread on its inner surface. The bolt 55 contains a screw thread on its predetermined portion at the frontal end, which interlocks with the screw thread on the hole (47). The head (56) is located on the rear side of the bolt 55, on which the user can press. A compression spring 57 is implemented around the bolt 55, and this bolt 55, penetrates through the hole 54 on the second coupling part 52 to combine with the hole 47 on the first coupling part 45, causing the toothed latch wheels 46, 53 to interlock with each other. One side of the hole 54 formed internal of the second coupling part 52 is formed with a diameter large enough to include the bolt 55 equipped with the compression spring 57. The other side of the hole 54, through which the bolt 55 is passing, is formed in a smaller diameter in order to provide enough support and pressure to compress upon the compression spring 57.

When the head 56 of the bolt 55 is pressed upon, the compression spring 57 in the second coupling part 52 is compressed and the first coupling part 45, which is combined with the bolt 55, is pushed away. As a result, the toothed latch wheel 46 of the first coupling part 45 is set apart from the toothed latch wheel 53 of the second coupling part 52.

Consequently, the chin-supporting unit 10 becomes available to swing downward in Direction 2 around the hinge units 30a, 30b as its axis, allowing to reduce the interval distance between the chin-supporting unit 10 and the shoulder-supporting unit 20. The user does not need to press onto the head 56 of the bolt 55 when desired to widen the interval distance between the chin-supporting unit 10 and the shoulder-supporting unit 20, because the toothed latch wheels 46, 53 can still rotate upward in Direction 1 while still interlocked with each other.

The upper regulatory arm 41 include the pinning body 42, where the first coupling part 45 is mounted on the bottom end, and the tension-providing body 43, which contains a spring 44 at its one end. This end of the tension-providing body 43 incorporating the internal spring 44 is inserted into the pinning body 42 to form a complex and exert tension on the chin-supporting unit 10. In order to prevent the tension-providing body 43 from easily separating from the complex, one end of the internal spring 44 may be attached on the inner wall of the pinning body 42.

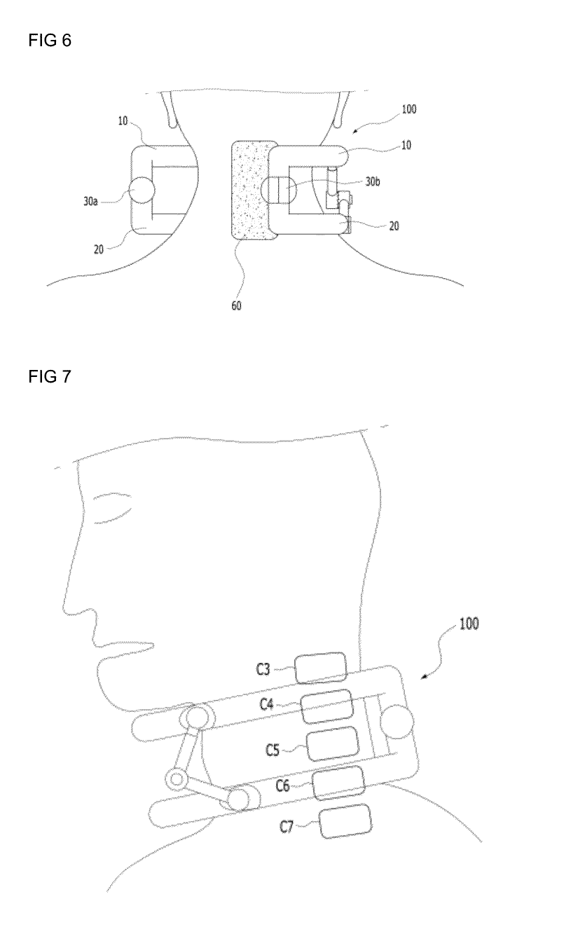

Referring to FIGS. 5 and 6, the lordosis-forming cervical brace 100 may also include the cervical-supporting unit 60.

The cervical-supporting unit 60 is located in the center of the rear bent portions of the chin-supporting unit 10 and the shoulder-supporting unit 20 and this plate-shaped cushion provides a stable support on the cervical vertebrae. Specifically, the cervical-supporting unit 60 should be attached to only one side of the rear bent portion so that the user may easily open up and take off the cervical brace 100.

FIG. 7 illustrates a patient, whose cervical vertebrae (C3-C7) has lost lordosis and become straightened, wearing the lordosis-forming cervical brace 100 around neck.

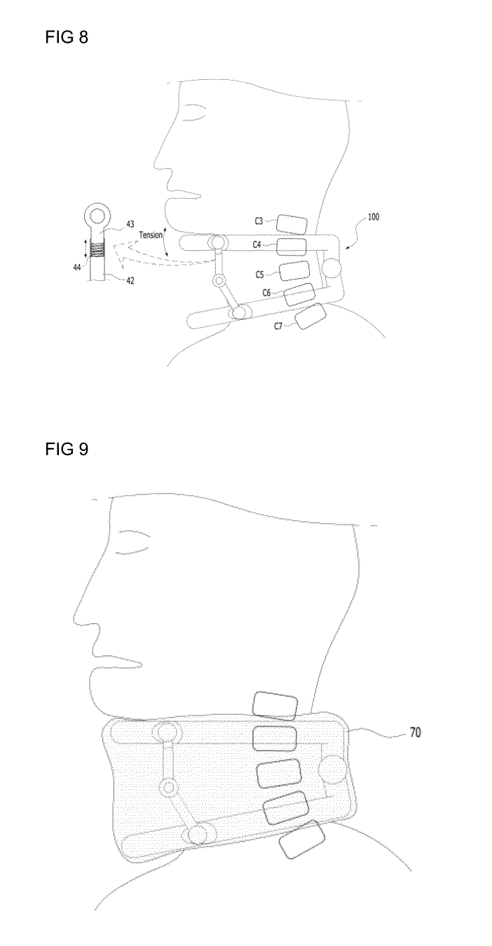

FIG. 8 illustrates the formation of lordosis on the cervical vertebrae (C3-C7) of a patient that is achieved by lifting the chin area of the patient, as the interval distance between the chin-supporting unit 10 and the shoulder-supporitng unit 20 increases, rotating around the hinge units 30a, 30b.

Moreover, while the chin-supporting unit 10 and the shoulder-supporting unit 20 maintain distance in between, the tension-providing unit 43 absorbs shock by providing tension in accordance with the movement of the chin, thus resulting in a comfortable fit of the invention.

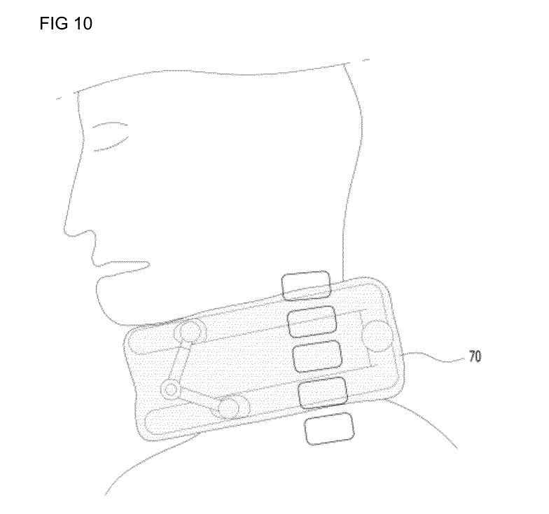

Referring to FIGS. 9 and 10, the lordosis-forming cervical brace 100 ideally fitted in the covering 70 for use.

The cervical brace 100 is equipped inside of the covering 70, which is made of a flexible material. In order to allow an easy operation of a user, the control units 40a, 40b may either be partially exposed external to the cover 70 or be tightly fitted to silhouette against the covering 70. Additionally, the covering 70 is ideally made of a well-ventilated and skin-friendly (non-irritating) material.

This invention of the lordosis-forming cervical brace is able to correct the shape of the cervical vertebrae by effectively forming its “C-shaped” forward curvature, or normal lordosis, instead of simply raising the chin.

Moreover, no complicated operation but only the simple lifting motion of the chin-supporting unit is necessary to fix it at a desired position to facilitate the formation of a normal lordosis on the cervical vertebrae. The chin-supporting unit can be reversed in its motion and lowered, by simply pressing on the buttons located on the both sides of the cervical brace.

The present invention is not limited to the preferred embodiment of the specifics noted above. Various modifications may be suggested by those skilled in the art, without departing from the scope of the present invention. All such changes and variations are intended to be within the scope of the present invention as defined by the appended claims.

1. A cervical brace for forming lordosis in cervical vertebrae comprising:
A circular ring-shaped chin-supporting unit;
A circular ring-shaped shoulder-supporting unit;
Two hinge units that connect the chin-supporting and shoulder-supporting units, equipped at both sides of the rear;
and two control units, located at both sides of the front, which maintain and adjust the gap distance between the two units at the front, connecting the two chin-supporting unit and shoulder-supporting unit,
wherein the frontal portion of the chin-supporting unit rotates upward around the hinge units, creates an interval distance between the chin-supporting unit and the shoulder-supporting unit, and comes to a halt once the above control units lock up and maintain the interval gap distance.

2. A cervical brace for forming lordosis in cervical vertebrae as defined in claim 1, wherein the control units are comprised of articulating parts, which include the interlocking toothed latch wheels, extend as the chin-supporting unit rotates upward, and lock up once the articulating parts interlock when the chin-supporting unit comes to a halt, thereby maintaining the internal distance between the chin-supporting unit and the shoulder-supporting unit, while pivoting in the opposite direction to allow the chin-supporting unit to rotate downward when the interlocked toothed latch wheels are separated from each other.

3. A cervical brace forming lordosis in cervical vertebrae as defined in claim 2, wherein the above control units are comprised of the upper regulatory arm and the lower regulatory arm, centering on the articulating parts, where either or both of the upper regulatory arm and lower regulatory arm contain internal springs to provide tension when the chin-supporting unit is bearing weight.

4. A cervical brace forming lordosis in cervical vertebrae as defined in claim 1, wherein the above-mentioned control units consist of the following:
the upper regulatory arm that can assemble and combine with the chin-supporting unit;
the first coupling part that is located at the bottom end of the upper regulatory arm and includes a toothed latch wheel;
the second coupling part of which toothed latch wheel interlocks with the toothed latch wheel of the first coupling part;
the lower regulatory arm that is located at the bottom end of the second coupling part and can assemble and combine with the shoulder-supporting unit;
the equipped bolt that penetrates the first coupling part and the second coupling part;
and the compression spring that is implemented around the penetrating bolt to interlock the first coupling part and the second coupling part.

5. A cervical brace forming lordosis in cervical vertebrae as defined in claim 4, wherein when the above head of the bolt is pressed upon, the toothed latch wheel of the first coupling part is separated from the toothed latch wheel of the second coupling part, thereby allowing the above-mentioned chin-supporting part to rotate downward around the hinge units as its axis.

6. A cervical brace forming lordosis in cervical vertebrae as defined in claim 4, wherein the aforementioned upper regulatory arm consist of the following:
the pinning body, where the first coupling part is mounted on its bottom end; and
the tension-providing body, which contains a spring at its one end, thereby this end of the tension-providing body incorporating the internal spring is inserted into the pinning body to form a complex and exert tension on the chin-supporting unit.

7. A cervical brace forming lordosis in cervical vertebrae as defined in claim 1, wherein the cervical-supporting unit, which is a plate-shaped cushion, is located in the center of the rear bent portions of the chin-supporting unit and the shoulder-supporting unit to provide a stable support on the cervical vertebrae.

8. A cervical brace forming lordosis in cervical vertebrae as defined in claim 1, wherein the cervical-supporting cushion is enclosed in a covering made out of flexible material, while the control units may either be partially exposed external to the cover or be tightly fitted to silhouette against the covering in order to allow an easy operation of the device.
